(19) United States
(12) Patent Application Publication
(10) Pub. No.: US 2014/0348848 A1
(43) Pub. Date: Nov. 27, 2014

(54) ANTI-IL-1BETA (INTERLEUKIN-1BETA) ANTIBODY-BASED PROPHYLACTIC THERAPY TO PREVENT COMPLICATIONS LEADING TO VASO-OCCLUSION IN SICKLE CELL DISEASE

(71) Applicant: Dhananjay Kaul, White Plains, NY (US)

(72) Inventor: Dhananjay Kaul, White Plains, NY (US)

(21) Appl. No.: 14/361,363

(22) PCT Filed: Nov. 29, 2012
(86) PCT No.: PCT/US2012/067057

§ 371 Date: May 29, 2014

Related U.S. Application Data
(60) Provisional application No. 61/566,130, filed on Dec. 2, 2011.

(51) Int. Cl. C07K 16/24

(52) U.S. Cl. CPC C07K 16/245; A61K 2039/505
USPC 424/145.1; 530/388.23

(57) ABSTRACT

This invention generally relates to a novel use of IL-1β-ligand/IL-1 receptor disrupting compounds (herein referred to also as “IL-1beta Compounds”) in a therapy preventing manifestations and especially complications leading to subsequent damages, such as vaso-occlusion, in individuals being threatened by sickle cell disease (that is, especially homozygous HbS gene carriers, heterozygotes with sickle-beta-thalassemia with an SCD supporting combination of HbS gene and beta-thal gene, an individual with one sickle cell gene and one null alleleor an individual with hemoglobin SC disease).

### SUMMARY OF THE INVENTION

This invention generally relates to a novel use of IL-1β-ligand/IL-1 receptor disrupting compounds (herein referred to also as “IL-1beta Compounds”) in a therapy preventing manifestations and especially complications leading to subsequent damages, such as vaso-occlusion, in individuals being threatened by sickle cell disease (that is, especially homozygous HbS gene carriers). This and related invention aspects are described and claimed in more detail below.

### BACKGROUND OF THE INVENTION

Sickle cell disease affects a considerable percentage of people that come from tropical or subtropical regions where malaria is or was common, or descendants thereof. It is a recessive autosomal genetic blood disorder, and presence of one sickle cell gene in a person enhances fitness against malaria. This has led to an evolutionary selection of people carrying one allele of the gene, in spite of the strong disadvantages for carriers of two alleles. Therefore, one third of the persons in Sub-Saharian Africa carry the gene.

Sickle cell disease affects, for example, approximately 100 000 individuals in the USA, mainly Afro-American children where one out of 500 born will have sickle cell anemia, a form of sickle cell disease (SCD). About 250 000 cases are born new world-wide.

In sickle cell disease, endothelial activation and leukocyte recruitment (caused by reperfusion injury, inflammatory stimuli, infection or hypersensitivity reactions) can lead to flow stasis, intravascular sickling and vaso-occlusion, a hallmark of this disease. Inflammation is a recognized feature of SCD. Reperfusion injury (caused by vaso-occlusive events) and elevated levels of inflammatory cytokines in this disease are implicated in endothelial activation and inflammation. In transgenic sickle mice, exaggerated inflammatory response to hypoxia-reoxygenation is characterized by enhanced endothelial oxidant generation and leukocyte recruitment in venules (Kaul and Hebbel, J. Clin Invest. 106, 411-420, 2000). While during the hypoxic phase intravascular sickling is enhanced, reperfusion will induce exaggerated inflammatory response and leukocyte recruitment, which will promote flow stasis (Turhan et al. Proc. Natl. Acad. Sci. USA 99(5). 3047-3051, 2002). Hypoxia also induces endothelial cells and leukocytes to express and secrete proinflammatory cytokines such as interleukin-1beta (IL-1β). Sickle patients show elevated levels of IL-1β, which may, in part, be attributed to recurring ischemia-reperfusion episodes IL-1β can up-regulate endothelial adhesion molecules via endothelial oxidant generation and activation of nuclear factor-κB (NF-κB). In SCD, IL-1β is implicated in endothelial activation, cell adhesion and inflammation (Natarajan et al., Blood 87, 4845-4852 (1996); Zachlederova and Jarolim, Blood Cells Mol. Dis. 30, 70-81, 2003; Wanderer, J. Pediatr. Hematol. Concol. 31(8), 537-538, 2009).

Elevated levels of inflammatory cytokines in SCD are implicated in endothelial activation and leukocyte recruitment that can potentially lead to painful vaso-occlusive crisis, as already indicated being a hallmark of this disease. An inflammatory condition in sickle cell anemia is further indicated by higher than normal peripheral leukocyte counts and increase in soluble endothelial adhesion molecules such as vascular cell adhesion molecule (sVCAM-1) and intercellular adhesion molecule-1 (sICAM-1). In SCD, infections are often followed by the occurrence of a vaso-occlusive crisis. Inflammatory conditions, infections or hypersensitivity reactions will cause abnormal activation of endothelium and increased leukocyte recruitment in postcapillary venules resulting in sluggish blood flow, increased microvascular transit times, hypoxia, red cell sickling and vaso-occlusion. Hence, therapeutic approaches are required to prevent such complications leading to vaso-occlusion in SCD.

It is a problem to be solved by the present invention to avoid the critical manifestations and complications leading to and caused by SCD.

We hypothesized that targeting IL-1β will allow to attenuate endothelial activation and exert anti-inflammatory effect in SCD.

### General Description of the Invention

Surprisingly, here for the first time the efficacy of anti-IL-1β therapy in alleviation of reperfusion injury, endothelial activation, inflammation and flow stasis caused by hypoxia-reoxygenation in transgenic sickle mice is shown, as well as its beneficial effect in normoxic homozygous sickle (BERK) mice. The results provide a strong basis for preventative application of this antibody in (e.g. clinical) management of SCD in humans.

It is a central aspect to the present invention that use of anti-IL-1β antibody allows to prevent complications (i.e., endothelial activation and leukocyte recruitment caused by inflammation, infection or hypersensitivity reactions) that can lead to intravascular sickling and vaso-occlusion in sickle cell disease.

Thus it is possible to allow to maintain good hemodynamics, to mitigate or inhibit the endothelial activation of lymphocytes and/or to allow to mitigate or inhibit attacks or crisis caused by intravascular occlusion.

Details of the Invention are described in the following:

### DETAILED DESCRIPTION OF THE INVENTION

The invention, in a first embodiment, relates to a method of using (=use of) a preventatively effective amount of an IL-1β-ligand/IL-1 receptor disrupting compound (herein referred to also as “IL-1beta Compound”) for the prevention of manifestations and especially complications, e.g. endothelial activation, leukocyte recruitment caused e.g. by inflammation, infection or hypersensitivity reactions, that can lead to intravascular sickling and vaso-occlusion in sickle cell disease in a patient in need of such use, especially an individual with homozygosity for the mutation that causes hemoglobin S (HbS), a heterozygote (meaning a heterocygotic individual whereever used herein) with sickle-beta-thalassemia with an SCD supporting combination of HbS gene and beta-thal gene, an individual with one sickle cell gene and one null allele or an individual with hemoglobin SC disease.

The present invention, in a further embodiment, also relates to an IL-1β-ligand/IL-1 receptor disrupting compound for use in a method as described above and below or generally in a method for the prevention of manifestations and especially complications, e.g. endothelial activation, leukocyte recruitment caused e.g. by inflammation, infection or hypersensitivity reactions, that can lead to intravascular sickling and vaso-occlusion in sickle cell disease (SCD) in a patient.

The present invention, in yet a further embodiment, also relates to the use of an IL-1β-ligand/IL-1 receptor disrupting compound for the prevention of manifestations and especially complications. e.g. endothelial activation, leukocyte recruitment caused e.g. by inflammation, infection or hypersensitivity reactions, that can lead to intravascular sickling and vaso-occlusion in sickle cell disease, especially in a patient in need of such use, especially an individual with homozygosity for the mutation that causes hemoglobin S (HbS), a heterozygote with sickle-beta-thalassemia with an SCD supporting combination of HbS gene and beta-thal gene, an individual with one sickle cell gene and one null alleleor an individual with hemoglobin SC disease.

The present in invention, in another embodiment, also relates to the use of an IL-1β-ligand/IL-1 receptor disrupting compound for the manufacture of a pharmaceutical formulation for the prevention of manifestations and especially complications, e.g. endothelial activation, leukocyte recruitment caused e.g. by inflammation, infection or hypersensitivity reactions, that can lead to intravascular sickling and vaso-occlusion in sickle cell disease, especially in a patient in need of such use (especially preventative/prophylactic treatment), especially an individual with homozygosity for the mutation that causes hemoglobin S (HbS), a heterozygote with sickle-beta-thalassemia with an SCD supporting combination of HbS gene and beta-thal gene, an individual with one sickle cell gene and one null alleleor an individual with hemoglobin SC disease.

In yet another embodiment, the present invention relates to a pharmaceutical formulation comprising an IL-1β-ligand/IL-1 receptor disrupting compound for use in a method of prevention as described above and below or generally in a method for the prevention of manifestations and especially complications, e.g. endothelial activation, leukocyte recruitment caused e.g. by inflammation, infection or hypersensitivity reactions, that can lead to intravascular sickling and vaso-occlusion in sickle cell disease in a patient.

An additional or alternative embodiment of the invention relates to a combination product comprising an IL-1β-ligand/IL-1 receptor disrupting compound and a co-agent, for simultaneous and/or timely staggered use in a method of use as described above.

The following definitions can replace (singly, by two or more or all) general expressions used above and below and thus define more specific invention embodiments. In the claims (which are incorporated here by reference), some of these and further invention embodiments are provided.

An IL-1β-ligand/IL-1 receptor disrupting compound (“IL-1beta Compound”) can be selected from small molecular compounds disrupting IL-1β ligand-IL-1 receptor interaction, IL-1β antibodies or IL-1 receptor antibodies. e.g. IL-1β binding molecules described herein, e.g. antibodies disclosed herein, e.g. IL-1β binding compounds or IL-1 receptor binding compounds, and/or RNA compounds decreasing either IL-1β ligands or IL-1 receptor protein levels.

A particular IL-1beta Compound used according to the invention is an IL-1β binding molecule which comprise an antigen binding site comprising at least one immunoglobulin heavy chain variable domain (V_{H}) which comprises in sequence hypervariable regions CDR1, CDR2 and CDR3, said CDR1 having the amino acid sequence Val-Tyr-Gly-Met-Asn (SEQ ID NO: 3), said CDR2 having the amino acid sequence Ile-Ile-Trp-Tyr-Asp-Gly-Asp-Asn-Gln-Tyr-Tyr-Ala-Asp-Ser-Val-Lys-Gly (SEQ ID NO: 4), and said CDR3 having the amino acid sequence Asp-Leu-Arg-Thr-Gly-Pro (SEQ ID NO: 5); and direct equivalents thereof.

In yet a further embodiment, an IL-1beta Compound used according to the invention is an IL-1β binding molecule which comprise at least one immunoglobulin light chain variable domain (V_{L}) which comprises in sequence hypervariable regions CDR1′, CDR2′ and CDR3′, said CDR1′ having the amino acid sequence Arg-Ala-Ser-Gln-Ser-Ile-Gly-Ser-Ser-Leu-His (SEQ ID NO: 6), said CDR2′ having the amino acid sequence Ala-Ser-Gln-Ser-Phe-Ser (SEQ ID NO: 7) and said CDR3′ having the amino acid sequence His-Gln-Ser-Ser-Ser-Leu-Pro (SEQ ID NO: 8); and direct equivalent thereof.

In yet a further embodiment, an IL-1beta Compound used according to the invention is a single domain IL-1beta binding molecule comprising an isolated immunoglobulin heavy chain comprising a heavy chain variable domain (V_{H}) as defined above.

In yet a further embodiment, an IL-1beta Compound used according to the invention is an IL-1β binding molecule comprising both heavy (V_{H}) and light chain (V_{L}) variable domains in which said IL-1β binding molecule comprises at least one antigen binding site comprising:
a) an immunoglobulin heavy chain variable domain (V_{H}) which comprises in sequence hypervariable regions CDR1, CDR2 and CDR3, said CDR1 having the amino acid sequence Val-Tyr-Gly-Met-Asn (SEQ ID NO: 3), said CDR2 having the amino acid sequence Ile-Ile-Trp-Tyr-Asp-Gly-Asp-Asn-Gln-Tyr-Tyr-Ala-Asp-Ser-Val-Lys-Gly (SEQ ID NO: 4), and said CDR3 having the amino acid sequence Asp-Leu-Arg-Thr-Gly-Pro (SEQ ID NO: 5), and
b) an immunoglobulin light chain variable domain (V_{L}) which comprises in sequence hypervariable regions CDR1′, CDR2′ and CDR3′, said CDR1′ having the amino acid sequence Arg-Ala-Ser-Gln-Ser-Ile-Gly-Ser-Ser-Leu-His (SEQ ID NO: 6), said CDR2′ having the amino acid sequence Ala-Ser-Gln-Ser-Phe-Ser (SEQ ID NO: 7), and said CDR3′ having the amino acid sequence His-Gln-Ser-Ser-Ser-Leu-Pro (SEQ ID NO: 8);
   and direct equivalents thereof.

Unless otherwise indicated, any polypeptide chain is herein described as having an amino acid sequence starting at the N-terminal extremity and ending at the C-terminal extremity.

When the antigen binding site comprises both the V_{H} and V_{L} domains, these may be located on the same polypeptide molecule or, preferably, each domain may be on a different chain, the V_{H} domain being part of an immunoglobulin heavy chain or fragment thereof and the V_{L} being part of an immunoglobulin light chain or fragment thereof.

By “IL-1β binding molecule” is meant any molecule capable of binding to the IL-1β ligand either alone or associated with other molecules. The binding reaction may be shown by standard methods (qualitative assays) including, for example, a bioassay for determining the inhibition of IL-1β binding to its receptor or any kind of binding assays, with reference to a negative control test in which an antibody of unrelated specificity but of the same isotype, e.g. an anti-CD25 antibody, is used. Advantageously, the binding of the IL-1β binding molecules of the invention to IL-1β may be shown in a competitive binding assay.

Examples of antigen binding molecules include antibodies as produced by B-cells or hybridomas and chimeric, CDR-grafted or human antibodies or any fragment thereof, e.g. F(ab′)₂ and Fab fragments, as well as single chain or single domain antibodies.

A single chain antibody consists of the variable domains of the heavy and light chains of an antibody covalently bound by a peptide linker usually consisting of from 10 to 30 amino acids, preferably from 15 to 25 amino acids. Therefore, such a structure does not include the constant part of the heavy and light chains and it is believed that the small peptide spacer should be less antigenic than a whole constant part. By “chimeric antibody” is meant an antibody in which the constant regions of heavy or light chains or both are of human origin while the variable domains of both heavy and light chains are of non-human (e.g. murine) origin or of human origin but derived from a different human antibody. By “CDR-grafted antibody” is meant an antibody in which the hypervariable regions (CDRs) are derived from a donor antibody, such as a nonhuman (e.g. murine) antibody or a different human antibody, while all or substantially all the other parts of the immunoglobulin e.g. the constant regions and the highly conserved parts of the variable domains, i.e. the framework regions, are derived from an acceptor antibody, e.g. an antibody of human origin. A CDR-grafted antibody may however contain a few amino acids of the donor sequence in the framework regions, for instance in the parts of the framework regions adjacent to the hypervariable regions. By “human antibody” is meant an antibody in which the constant and variable regions of both the heavy and light chains are all of human origin, or substantially identical to sequences of human origin, not necessarily from the same antibody and includes antibodies produced by mice in which the murine immunoglobulin variable and constant part genes have been replaced by their human counterparts. e.g. as described in general terms in EP 0546073 B1, U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, EP 0 438474 B1 and EP 0 463151 B1.

Particularly preferred IL-1β binding molecules of the invention are monoclonal antibodies, especially the ACZ 885 antibody (canakinumab) as hereinafter described in the Examples and in WO 02/16436.

Other IL-1β binding molecules that can be used in conjunction with the invention are the monoclonal antibodies like. Xoma-052, gevokizumab, LY-2189102 or AMG-108.

Thus in preferred antibodies of the invention, the variable domains of both heavy and light chains are of human origin, for instance those of the ACZ 885 antibody (canakinumab) which are shown in SEQ ID NO:1 and SEQ ID NO:2. The constant region domains preferably also comprise suitable human constant region domains, for instance as described in “Sequences of Proteins of Immunological Interest”. Kabat E. A. et al, US Department of Health and Human Services. Public Health Service, National Institute of Health.

Hypervariable regions may be associated with any kind of framework regions, though preferably are of human origin. Suitable framework regions are described in Kabat E. A. et al, ibid. The preferred heavy chain framework is a human heavy chain framework, for instance that of the ACZ 885 antibody which is shown in SEQ ID NO:1. It consists in sequence of FR1, FR2, FR3 and FR4 regions. In a similar manner, SEQ ID NO:2 shows the preferred ACZ 885 light chain framework which consists, in sequence, of FR1′, FR2′, FR3′ and FR4′ regions.

Accordingly, the invention also provides an IL-1β binding molecule which comprises at least one antigen binding site comprising either a first domain having an amino acid sequence substantially identical to that shown in SEQ ID NO:1 starting with the amino acid at position 1 and ending with the amino acid at position 118 or a first domain as described above and a second domain having an amino acid sequence substantially identical to that shown in SEQ ID NO:2, starting with the amino acid at position 1 and ending with the amino acid at position 107.

Monoclonal antibodies raised against a protein naturally found in all humans are typically developed in a non-human system e.g. in mice, and as such are typically non-human proteins. As a direct consequence of this, a xenogenic antibody as produced by a hybridoma, when administered to humans, elicits an undesirable immune response which is predominantly mediated by the constant part of the xenogenic immunoglobulin. This clearly limits the use of such antibodies as they cannot be administered over a prolonged period of time. Therefore it is particularly preferred to use single chain, single domain, chimeric, CDR-grafted, or especially human antibodies which are not likely to elicit a substantial allogenic response when administered to humans.

In view of the foregoing, a more preferred IL-1β binding molecule of the invention is selected from a human anti IL-1β antibody which comprises at least
a) an immunoglobulin heavy chain or fragment thereof which comprises (i) a variable domain comprising in sequence the hypervariable regions CDR1, CDR2 and CDR3 and (ii) the constant part or fragment thereof of a human heavy chain; said CDR1 having the amino acid sequence Val-Tyr-Gly-Met-Asn (SEQ ID NO: 3), said CDR2 having the amino acid sequence Ile-Ile-Trp-Tyr-Asp-Gly-Asp-Asn-Gln-Tyr-Tyr-Ala-Asp-Ser-Val-Lys-Gly (SEQ ID NO: 4), and said CDR3 having the amino acid sequence Asp-Leu-Arg-Thr-Gly-Pro (SEQ ID NO: 5) and
b) an immunoglobulin light chain or fragment thereof which comprises (i) a variable domain comprising in sequence the hypervariable regions and optionally also the CDR1′. CDR2′, and CDR3′ hypervariable regions and (ii) the constant part or fragment thereof of a human light chain, said CDR1′ having the amino acid sequence Arg-Ala-Ser-Gln-Ser-Ile-Gly-Ser-Ser-Leu-His (SEQ ID NO: 6), said CDR2′ having the amino acid sequence Ala-Ser-Gln-Ser-Phe-Ser (SEQ ID NO: 7), and said CDR3′ having the amino acid sequence His-Gln-Ser-Ser-Ser-Leu-Pro (SEQ ID NO: 8);
   and/or direct equivalents thereof.

Alternatively, an IL-1β binding molecule of the invention may be selected from a single chain binding molecule which comprises an antigen binding site comprising
a) a first domain comprising in sequence the hypervariable regions CDR1, CDR2 and CDR3, said CDR1 having the amino acid sequence Val-Tyr-Gly-Met-Asn (SEQ ID NO: 9), said CDR2 having the amino acid sequence Ile-Ile-Trp-Tyr-Asp-Gly-Asp-Asn-Gln-TyrTyr-Ala-Asp-Ser-Val-Lys-Gly (SEQ ID NO: 4), and said CDR3 having the amino acid sequence Asp-Leu-Arg-Thr-Gly-Pro (SEQ ID NO: 5).
b) A second domain comprising the hypervariable regions CDR1′, CDR2′ and CDR3′, said CDR1′ having the amino acid sequence Arg-Ala-Ser-Gln-Ser-Ile-Gly-Ser-Ser-Leu-His (SEQ ID NO: 6), said CDR2′ having the amino acid sequence Ala-Ser-Gln-Ser-Phe-Ser (SEQ ID NO: 7), and said CDR3′ having the amino acid sequence His-Gln-Ser-Ser-Ser-Leu-Pro (SEQ ID NO: 8), and
c) a peptide linker which is bound either to the N-terminal extremity of the first domain and to the C-terminal extremity of the second domain or to the C-terminal extremity of the first domain and to the N-terminal extremity of second domain;
   and/or direct equivalents thereof.

As it is well known, minor changes in an amino acid sequence such as deletion, addition or substitution of one, a few or even several amino acids may lead to an allelic form of the original protein which has substantially identical properties.

Thus, by the term “direct equivalents thereof” is meant either any single domain IL-1β binding molecule (molecule X)
(i) in which the hypervariable regions CDR1, CDR2 and CDR3 taken as a whole are at least 80% homologous, preferably at least 90% homologous, more preferably at least 95% homologous to the hypervariable regions as shown above and,
(ii) which is capable of inhibiting the binding of IL-1β to its receptor substantially to the same extent as a reference molecule having framework regions identical to those of molecule X but having hypervariable regions CDR1, CDR2 and CDR3 identical to those shown in above.
   or any IL-β binding molecule having at least two domains per binding site (molecule X′)
(i) in which the hypervariable regions CDR1, CDR2, CDR3, CDR1′, CDR2′ and CDR3′ taken as a whole are at least 80% homologous, preferably at least 90% homologous, more preferably at least 95, 98 or 99% homologous, to the hypervariable regions as shown above and
(ii) which is capable of inhibiting the binding of IL-1β to its receptor substantially to the same extent as a reference molecule having framework regions and constant parts identical to molecule X′, but having hypervariable regions CDR1, CDR2, CDR3, CDR1′, CDR2′ and CDR3′, identical to those shown above.

In a further aspect the invention also provides an IL-1beta binding molecule comprising both heavy (V_{H}) and light chain (V_{L}) variable domains in which said IL-1beta binding molecule comprises at least one antigen binding site comprising:
a) an immunoglobulin heavy chain variable domain (V_{H}) which comprises in sequence hypervariable regions CDR1, CDR2 and CDR3, said CDR1 having the amino acid sequence Ser-Tyr-Trp-Ile-Gly (SEQ ID NO: 10), said CDR2 having the amino acid sequence Ile-Ile-Tyr-Pro-Ser-Asp-Ser-Asp-Thr-Arg-Tyr-Ser-Pro-Ser-Phe-Gn-Gly (SEQ ID NO: 11), and said CDR3 having the amino acid sequence Tyr-Thr-Asn-Trp-Asp-Ala-Phe-Asp-Ile (SEQ ID NO: 12), and
b) an immunoglobulin light chain variable domain (V_{L}) which comprises a CDR3′ hypervariable region having the amino acid sequence Gln-Gln-Arg-Ser-Asn-Trp-Met-Phe-Pro (SEQ ID NO: 13);
   and/or direct equivalents thereof.

In further aspect the invention provides an IL-1beta binding molecule comprising both heavy (V_{H}) and light (V_{L}) chain variable domains in which said IL-1beta binding molecule comprises at least one antigen binding site comprising:
a) an immunoglobulin heavy chain variable domain (V_{H}) which comprises in sequence hypervariable regions CDR1, CDR2 and CDR3, said CDR1 having the amino acid sequence Ser-Tyr-Trp-Ile-Gly (SEQ ID NO: 10), said CDR2 having the amino acid sequence Ile-Ile-Tyr-Pro-Ser-Asp-Ser-Asp-Thr-Arg-Tyr-Ser-Pro-Ser-Phe-Gln-Gly (SEQ ID NO: 12), and said CDR3 having the amino acid sequence Tyr-Thr-Asn-Trp-Asp-Ala-Phe-Asp-Ile (SEQ ID NO: 13), and
b) an immunoglobulin light chain variable domain (V_{L}) which comprises in sequence hypervariable regions CDR1′, CDR2′ and CDR3′, said CDR1′ having the amino acid sequence Arg-Ala-Ser-Gln-Ser-Val-Ser-Ser-Tyr-Leu Ala (SEQ ID NO: 14), said CDR2′ having the amino acid sequence Asp-Ala-Ser-Asn-Arg-Ala-Thr (SEQ ID NO: 15), and said CDR3′ having the amino acid sequence Gln-Gln-Arg-Ser-Asn-Trp-Met-Phe-Pro; (SEQ ID NO: 13)
   and/or direct equivalents thereof.

The inhibition of the binding of IL-1β to its receptor may be conveniently tested in various assays including such assays are described in WO 02/16436. By the term “to the same extent” is meant that the reference and the equivalent molecules exhibit, on a statistical basis, essentially identical IL-1β binding inhibition curves in one of the assays referred to above. For example, in IL-1β binding molecules of the invention typically have IC₅₀s for the inhibition of the binding of IL-1β to its receptor which are within +/−×5 of that of preferably substantially the same as, the IC₅₀ of the corresponding reference molecule when assayed as described above.

For example, the assay used may be an assay of competitive inhibition of binding of IL-1β by soluble IL-1 receptors and the IL-1β binding molecules of the invention.

Most preferably, the IL-1β binding molecule for use according to the invention is an human IL-1 antibody which comprises at least
a) one heavy chain which comprises a variable domain having an amino acid sequence substantially identical to that shown in SEQ ID NO:1 starting with the amino acid at position 1 and ending with the amino acid at position 118 and the constant part of a human heavy chain; and
b) one light chain which comprises a variable domain having an amino acid sequence substantially identical to that shown in SEQ ID NO:2 starting with the amino acid at position I and ending with the amino acid at position 107 and the constant part of a human light chain.

Most preferably, the IL-1β binding molecule for use according to the invention is canakinumab (also known as ACZ885, see Example).

The constant part of a human heavy chain may be of the γ₁, γ₂, γ₃, γ₄, μ, α₁, α₂, δ or ε type, preferably of the γ type, more preferably of the γ₁ type, whereas the constant part of a human light chain may be of the κ or λ type (which includes the λ₁, λ₂ and λ₃ subtypes) but is preferably of the κ type. The amino acid sequences of all these constant parts are given in Kabat et al ibid.

An IL-1β binding molecule useful according to the invention may be produced by recombinant DNA techniques as e.g. described in WO 02/16436.

In yet other variants of the invention embodiments, IL-1beta binding molecules (Compounds) may be antibodies which have binding specificity for the antigenic epitope of human IL-1β which includes the loop comprising the Glu 64 residue of mature human IL-1β (Residue Glu 64 of mature human IL-1β□ correspond to residue 180 of the human IL-1beta □precursor). This epitope is outside the recognition site of the IL-1beta □receptor and it is therefore most surprising that antibodies to this epitope, e.g. the ACZ 885 antibody (canakinumab), are capable of inhibiting the binding of IL-1β to its receptor.

For the purposes of the present description an antibody is “capable of inhibiting the binding of IL-1β” if the antibody is capable of inhibiting the binding of IL-β to its receptor substantially to the same extent as the ACZ 885 antibody, i.e. has a dissociation equilibrium constant (K_{D}) measured e.g. in a standard BIAcore analysis as disclosed in the Example of 10 nM or lower, e.g. in M or lower, preferably 100 pM or lower, more preferably 50 pM or lower.

Thus in a yet further aspect the invention provides the use of an IL-1β-ligand/IL-1 receptor disrupting compound, especially an antibody to IL-1β, which has a K_{D} for binding to IL-1β of about 10 nM, 1 nM, preferably 100 pM, more preferably 50 pM or less. This aspect of the invention also includes uses methods and compositions for such high affinity antibodies, as described above for antibodies to IL-1β have binding specificity for an antigenic determinant of mature human IL-1β which includes the loop comprising Glu 64.

In the present description amino acid sequences are at least 80%, 82%, 84%, 86%, 88%, 90%, 92%, 94%, 96% or 98% homologous to one another if they have at least 80%, 82%, 84%, 86%, 88%, 90%, 92%, 94%, 96% or 98% identical amino acid residues in a like position when the sequence are aligned optimally, gaps or insertions in the amino acid sequences being counted as non-identical residues.

“Homology” or “homologous” (or “identity” or “identical”) with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. Methods and computer programs for the alignment are well known.

Preferably, as used herein, the percent homology between two amino acid sequences or two nucleotide sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm as described in the following:

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al., 1990 J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., 1997 Nucleic Acids Res. 25(17):3389-3402. When utilizing BLAST and Gapped BLAST programs, the default parameters of the respective programs (e.g., XBLAST and NBLAST) can be used. See http:www.ncbi.nhn.nih.gov.

The phrase “direct equivalents” especially encompasses any (at least one, respectively) molecule, antibody or functional fragment thereof having the properties of an IL-1 beta binding molecule of the invention as provided in this description, including antibodies of various species and isotypes, Fab2, Fab and scFv fragments and mutants comprising 1, 2, 3, 4, 5, 6, 7, 8, 9, 10 or more point mutations in the CDR regions or outside the CDR regions of SEQ ID No's 1 and 2, especially 2 or in particular 1 point mutations leading to 1 or 2 substituted amino acids or to addition or insertion or deletion of especially 2 or in particular 1 amino acids in a specific sequence given herein, respectively.

The IL-1beta Compounds may be administered as the sole active ingredient or in conjunction with, e.g. as an adjuvant to, one or more other drugs (co-agent(s)) e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent, or with agents enhancing hemodynamics.

The IL-1beta compounds according to the invention may be administered as the sole active ingredient or in conjunction with a co-agent in sequential or simultaneous manner, in the form of a kit of parts or combined in one formulation.

For example, the IL-1beta Compounds, according to the invention, may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578. AP23573, AP23464, AP23675, AP23841, TAFA-93, biolimus-7 or biolimus-9; an ascomycin having immunosuppressive properties, e.g. ABT-281, ASM981, etc.; corticosteroids; cyclophosphamide; azathioprene; methotrexate: leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide □-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8. CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y: adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent. Immunomodulatory drugs which are prone to be useful in combination with a compound of the present invention include e.g.
mediators, e.g. inhibitors, of mTOR activity, including rapamycin of formula

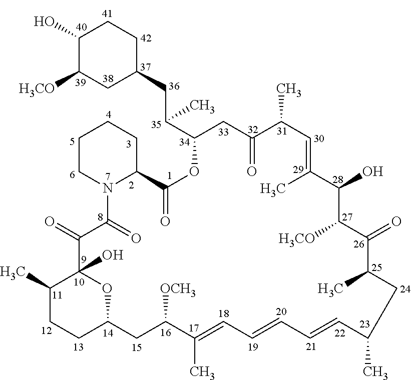 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

and rapamycin derivatives, e.g. including
40-O-alkyl-rapamycin derivatives, such as 40-O-hydroxyalkyl-rapamycin derivatives, such as 40-O-(2-hydroxy)-ethyl-rapamycin (everolimus),
32-deoxo-rapamycin derivatives and 32-hydroxy-rapamycin derivatives, such as 32-deoxorapamycin,
16-O-substituted rapamycin derivatives such as 16-pent-2-ynyloxy-32-deoxorapamycin, 16-pent-2-ynyloxy-32 (S or R)-dihydro-rapamycin. 16-pent-2-ynyloxy-32(S or R)-dihydro-40-O-(2-hydroxyethyl)-rapamycin.
rapamycin derivatives which are acylated at the oxygen group in position 40, e.g. 40-[3-hydroxy-2-(hydroxy-methyl)-2-methylpropanoate]-rapamycin (also known as CCI779),
rapamycin derivatives which are substituted in 40 position by heterocyclyl. e.g. 40-epi-(tetrazolyl)-rapamycin (also known as ABT578),
the so-called rapalogs. e.g. as disclosed in WO9802441, WO0114387 and WO0364383, such as AP23573, and/or
compounds disclosed under the name TAFA-93 and/or biolimus (biolimus A9).

IL-1beta compounds may advantageously be combined with Protein Kinase C Inhibitors and/or suppressors of T cell activation, in particular indolylmaleimide derivatives such as sotrastaurin (3-(1.H.-indol-3-yl)-4-[2-(4-methyl-piperazin-1-yl)-quinazolin-4-yl]-pyrrole-2,5-dione), for instance in order to inhibit the adaptive immune system and thereby further enhancing the therapeutic effects of IL-1beta compounds.

IL-1beta compounds may also advantageously be combined with anti-cytokines and/or anti-interleukins, in particular IL-17 binding compounds disclosed in WO2006/013107, optionally in combination with sotrastaurin.

IL-1beta compounds may advantageously be combined with anti TNFalpha compounds such as etanercept, adalimumab, infliximab, for instance in the treatment of RA and other (auto)-inflammatory diseases.

Among the agents enhancing hemodynamics, anticoagulants, such as warfarin, acenocoumarol, phenprocoumon, phenindione, heparin, fondaparinux, idraparinux, hirudin, lepirudin, bivalirudin, argatroban, dabigatran, ximelagatran, batroxobin or hementin, blood flow enhancers, such as chlofluperol, trifluperol, droperidol, metaclopramide, loxapine or butoxamine, may be mentioned.

Especially preferred as combination partners are other co-agents for treatment of SCD, such as inducers of expression of fetal hemoglobin, e.g. hydroxyurea, 5-azacytidine, erythropoietine, butyrates, e.g. sodium butyrate or arginine butyrate, or acetates; inhibitors of hemoglobin polymerization (sickling), such as nitric oxide or drugs delivering nitric oxide; reducers of intracellular hemoglobin concentrations, e.g. K—Cl-contransporter inhibitors or Ca-dependent potassium channel inhibitors, e.g. clotrimauzole or seniapoc, or Magnesium salts. e.g. MG pidolate; or other drugs, e.g. Nicosan.

Also analgesics and narcotics are among the preferred combination partners, as they allow for relief of pain associated with SCD. Examples of possible analgesics or narcotics as combination partners are NSAIDs such as salicylates, such as acetylsalicylic acid, diflunisal or salsalate, p-aminophenol derivatives, such as paracetamol or phenacetin, propionic acid derivatives, such as ibuprofen, naproxen, fenoprofen, ketoprofen, flurbiprofen, oxaprocin or loxoprofen, acetic acid derivatives, such as indomethacin, sulindac, etodolac, ketorolac, diclofenac or nabumetone, enolic acid (oxicam) derivatives, such as piroxicam, meloxicam, tenoxicam, droxicam, lornoxicam or isoxicam, fenamic acid derivatives, such as mefenamic acid, meclofenamic acid, flufenamic acid or tolfenamic acid, selective COX-2 inhibitors, such as celecoxib, rofecoxib, valdecoxib, parecoxib, lumiracoxib, etorixicib or firocoxib, sulphonanilides, such as nimesulide, or others, such as licofelone, flurpitine, opiates or opioids, such as morphine, dextromorphan, codeine, oxycodone, hydrocodone, dihydromorphine, pethidine buprenorphine, tramadol or venlafaxine, tricyclic antidepressants, such as amitriptyline, carbamazepine, gabapentin, pregabalin, psychotropic analgesic agents, e.g. cannabinoids, such as tetrahydrocannabinol, ketamine, clonidine or mexiletine, orphenadrine, cyclobenzaprine, scopolamine, atropine, gabapentin, methadone, ketobemidone, piritramide, or the like.

Among the manifestations and especially complications of SCD that can be subject to a preventative (prophylactical) therapy according to the invention, e.g. endothelial activation, leukocyte recruitment caused e.g. by inflammation, infection or hypersensitivity reactions (especially leading to leukocyte recruitment/leukocyte activation (especially recruitment of neutrophiles and neutrophilic inflammation)), are to be mentioned specifically, or also ischemia reperfusion injury, acute chest syndrome, especially acute chest injury.

Long term administration of an IL-1β-ligand/IL-1 receptor disrupting compound may also help avoid or reduce or delay the manifestation of asplenism or its precursor form hyposplenism, acute chest syndrome, cardiovascular damages or other manifestations of long term SCD, especially where caused by inflammation, infection or hypersensitivity reactions.

A patient in need of treatment is especially an individual with homozygosity for the mutation that causes hemoglobin S (HbS), e.g. in one particular variant of the invention embodiments a juvenile person or a child where SCD has not yet shown, or an adult where first or recurrent manifestations and especially complications by SCD are to be avoided.

For all indications disclosed herein this specifications (Indications of the inventions), the appropriate dosage will, of course, vary depending upon, for example, the particular IL-1beta Compounds, e.g. the Antibody of the Invention to be employed, the host, the mode of administration and the nature and severity of the condition being treated. However, in prophylactic use, satisfactory results are generally indicated to be obtained at dosages from about 0.05 mg to about 10 mg per kilogram body weight more usually from about 0.1 mg to about 5 mg per kilogram body weight. Antibody of the Invention is conveniently administered parenterally, intravenously, e.g. into the antecubital or other peripheral vein, intramuscularly, or subcutaneously. Advantageously, the dosages applied to an adult of 70 kg vary in the range from 1 mg to 400 mg per dosing, e.g. from 10 to 300 mg, 20 to 200 mg, 25 to 180 mg. 30 to 160 mg or 100 to 150 mg per dosing, respectively (with proportional variation in case of different weight being possible). For children, lower dosages (e.g. taking into account the difference in weight to an adult, different rates of metabolism and the like) may be possible, e.g. from 1 or 5 mg to 200 mg, 10 mg to 180 mg, 15 mg to 150 mg, 20 mg to 140 mg, 25 mg to 130 mg or 30 mg to 120 mg per dosing, respectively. For example, for adults fixed single doses of 20, 25, 50, 75, 100, 125, 150, 175, 200, 225, 250, 275 or 300 mg, respectively, may be employed, for children e.g. 5, 10, 20, 30, 50, 70, 80, 100, 125, or 150 mg, respectively.

In yet another embodiment, the invention concerns a surprising frequency of dosing for therapeutic uses, that is, the treatment schedule with IL-1beta Compounds, preferably IL-1beta antibodies, more preferably canakinumab, may consist in administration once every week or less frequently, more preferably once every 2 weeks or less frequently, more preferably once every 3 weeks or less frequently, more preferably once every month or less frequently, more preferably once every 2 months or less frequently, more preferably once every 3 months or less frequently, even more preferably once every 4 months or less frequently, even more preferably once every 5 months or less frequently, or even more preferably once every 6 months or less frequently. Most preferred is once every month or every third or every sixth month. In addition, any combination of the above mentioned dosing frequency can be used. For instance can a more frequent dosing according to the above be decreased to a less frequent dosing during the course of administration or vice versa.

Pharmaceutical compositions comprising an IL-1beta Compound useful according to the invention may be manufactured in conventional manner. A composition according to the invention is preferably provided in pulverized or especially lyophilized form. For immediate administration it is dissolved in a suitable aqueous carrier, for example sterile water for injection or sterile buffered physiological saline. Also liquid formulations e.g. in prefilled syringes or injectors are preferred. In the case of combinations, the combination partners may be present in separate compartments, or where both are for injection they may also be comprised in double- or multichamber injection devices, e.g. two- or multichamber syringes. A preferred formulation of antibodies for use according to the invention, especially of canakinumab, is disclosed in WO 2010/066762 which is incorporated here by reference.

If it is considered desirable to make up a solution of larger volume for administration by infusion rather as a bolus injection, it is possible and may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a possible suitable concentration is from 0.5 to 4.5% by weight of the saline solution.

The corresponding formulations are also part of the invention.

The IL-1beta Compound useful according to the invention are, for example, present or formulated to be present in the corresponding formulations, in each dosage from as administered (that is, after addition of carrier), in a weight share of 0.05 to 50 weight-%, such as 0.1 to 40, 1 to 30, 2 to 20, 2.5 to 18, 3 to 16 or 10 to 15 weight-%, respectively, or in pediatric formulations from 0.1 to 40, 0.1 to 20, 0.5 to 20, 1 to 18, 1.5 to 15, 2 to 14, 2.5 to 13 or 3 to 12 weight-%, respectively, in each case with respect to the total weight of all formulation compounds (IL-1beta Compound and carriers).

Co-agents (which may be present in the form of pharmaceutically acceptable salts or in non-salt form) may be formulated as customary, using one or more customary pharmaceutical carrier materials as appropriate. They may be present in customary amounts, e.g. from 1 to 98 weight percent of the respective formulation(s), and are administered in customary dosages, e.g. from 0.1 to 2000 mg per day and individual.

### DESCRIPTION OF THE FIGURES

The following Figure descriptions are also part of the disclosure of the embodiments of the present invention, especially complementing Example 1:

FIG. 1. Anti-IL-1β antibody (01BSUR, Novartis) ameliorates increased leukocyte adhesion in venules of sickle (NY1DD) mice caused by hypoxia-reoxygenation. NY1DD mice, untreated or receiving control isotype antibody and subjected to hypoxia-reoxygenation, showed pronounced 6- to 7.7-fold increases in leukocyte adhesion compared with normoxic NY1DD mice. In contrast, NY1DD mice receiving a single i.p. injection of anti-human IL-1β antibody prior to hypoxia showed markedly alleviated leukocyte adhesion; the resulting adhesion was not significantly different from that observed in normoxic C57BL and NY1DD mice. *P<0.026 vs. normoxic control C57BL; ⁺P<0.0001 vs. normoxic NY1DD mice; ^{#}P<0.0001 vs. NY1DD mice subjected to hypoxia-reoxygenation (untreated or receiving control isotype antibody). (Wilcoxon two-sample test).

FIG. 2. Anti-IL-1β antibody (01BSUR, Novartis) ameliorates increased leukocyte emigration from venules caused by hypoxia-reoxygenation in sickle (NY1DD) mice (untreated or receiving control isotype antibody). In contrast, NY1DD mice receiving a single i.p. injection of anti-IL-1β antibody prior to hypoxia showed marked alleviation of leukocyte emigration compared with NY1DD mice; the resulting leukocyte emigration was not significantly different from that observed in normoxic C57BL and NY1DD sickle mice. *P<0.0001 vs. normoxic NY1DD mice; ⁺P<0.0001 vs. NY1DD mice subjected to hypoxia-reoxygenation (untreated or receiving control isotype antibody). (Wilcoxon two-sample test).

FIG. 3. Representative fluorographs of cremaster venules in NY1DD sickle mice (top) and their corresponding DHR fluorescence profiles (bottom). (A) Normoxic NY1DD; (B) After 16 hr of hypoxia and 30 min of reoxygenation: (C) Effect of pretreatment of NY1DD mice with anti-IL-1β antibody followed by hypoxia-reoxygenation. Under normoxic conditions, there was little or no evidence of oxidant generation in venular endothelium. Note the marked increase in DHR fluorescence after hypoxia-reoxygenation. Pretreatment with anti-IL-1β antibody almost completely attenuated endothelial oxidant generation after hypoxia-reoxygenation.

FIG. 4. Hypoxia-reoxygenation (H-R) in NY1DD mice: the effect of pretreatment with anti-IL-1b antibody on the relative intensity (Δ/) of DHR fluorescence in venular endothelium. Δ/ showed a marked increase with reoxygenation compared with normoxic mice. Marked decrease in DHR oxidation was evident after pretreatment with anti-IL-1β antibody. *P<0.00001 vs. normoxic NY1DD mice; ^{#}P<0.00001 vs. H-R alone.

FIG. 5. Serum sVCAM-1 concentrations showed a significant decrease in NY1DD mice infused with anti-IL-1β antibody before hypoxia-reoxygenation. *P<0.05 vs. Normoxic NY1DD; ⁺P<0.003 vs. untreated NY1DD mice subjected to hypoxia-reoxygenation.

### EXAMPLES

The following Examples illustrate the invention without limiting its scope:

Note that instead of the antibodies used comparable antibodies can be used and are obtainable from various sources. For example, regarding the control antibody used, monoclonal control antibodies, e.g. against mouse anti-cyclosporin A, are obtainable from various companies, Genway Biotech, Inc. (San Diego, Calif., USA). Novus Biologicals, LLC (Littleton, Colo., USA), AbD Serotec (Kidlington, UK), LifeSpan Biosciences (Seattle, Wash., USA), Raybiotech, Inc. (Norcross, Ga., USA), Acris Antibodies GmbH (Herford. Germany), and others.

### Example 1

### Efficacy of a Monoclonal Antibody 01BSUR to Interleukin-1β□ (IL-1β) in the Prevention of SCD Manifestations and Complications

### Approach and Methods:

In our studies, we subjected transgenic sickle (NY1DD) mice (see above and Fabry et al., Proc. Natl. Acad. Sci (USA) 89, 12150-12154 (1992)) to 16 hr of hypoxia (8% O₂, 0.5% CO₂, balance N₂) followed by 3 hr of reoxygenation at ambient air. We selected NY1DD mice based on their highly exaggerated response to hypoxia-reoxygenation compared with C57BL (control) mice as reported in our previous studies (Kaul, D K and Hebbel. R P, J Clin Invest 106:411-420, 2000). NY1DD mice have been extensively backcrossed onto C57BL background.

NY1DD mice express approximately 56% human α and approximately 75% β^{S} (of all β globin) on a mouse homozygous β^{mejor} deletional background (Fabry et al., Proc. Natl. Acad. Sci (USA) 89, 12150-12154 (1992)).

The following groups of mice were tested for leukocyte adhesion, leukocyte emigration (extravasation), and microvascular flow parameters (i.e. red cell velocity [Vrbc], wall shear rates and volumetric flow [Q]). Direct microscopic observations and videotaping were carried out to determine leukocyte adhesion in venules and emigration from venules, the sites of inflammatory response. To quantify leukocyte adhesion in venules, we measured adherent leukocytes (per 100 μm length of the vessels) and emigrated leukocytes (no. of egressed leukocytes adjacent to venules) as previously described in our in vivo studies (Kaul et al. Am J Physiol Heart Circ Physiol 287:H293-H301, 2004; Kaul and Hebbel, J Clin Invest 106.411-420, 2000). Intravital measurements in the cremaster microcirculation included on-line measurements of vessel diameter using a video image shearing device and red cell velocity (Vrbc) using a dual-slit photodiode and cross-correlator as described (Silva and Intaglietta, Microvasc Res 7:156-169, 1974; Wayland and Johnson, J Appl Physiol 22:333-337, 1967). Wall shear rates and volumetric flow rates (Q) were calculated from vessel diameters and centerline Vrbc (Baker and Wayland, Microvasc Res 7:131-143, 1974; Lipowsky et al. Microvasc Res 19:297-319, 1980).

1) Normoxic wild type (C57BL) mice (n=5);
2) Normoxic sickle (NY1DD) mice (n=9);
3) NY1DD Sickle mice (n=8) subjected to hypoxia-reoxygenation (untreated); and
4) NY1DD mice (n=6) infused with 200 μg of Novartis murine anti-IL-1β antibody used in order to avoid cross-reaction issues, said antibody having demonstrated activity in vivo (surrogate antibody named 01BSUR: an analogous antibody with a murine IgG2a/k isotype that recognizes the intended antigen in different species but does not cross-react with the human antigen and also not with IL-1 and IL-1Ra; for details on the antibody see Geiger et al, Clin. Exp. Rheumatol. 11, 515-522 (1993) and Williams et al., J. Immunol. 165, 7240-7245 (2000); see also (http:/Iwww.ema.europa.eu/docs/en_GB/document_library/EPAR_Public_assessment_report/human/001109/WC500031679.pdf)).

For intraperitoneal injection in mice, for each mouse 200 μg antibody (control or 01BSUR) was formulated in 0.2 ml sterile phosphate buffered saline (PBS, pH 7.4).

To test the specificity of the test antibody, NY1DD mice (n=2) were infused with 200 μg of control isotype antibody (monoclonal anti-cyclosporin A antibody, a mouse IgG2a). Antibodies were infused i.p. 24 hrs before the onset of hypoxia. Thereafter, the cremaster muscle microvasculature was exposed and microcirculatory parameters and endothelial oxidant generation measured in venules, which are the sites of inflammatory response. The cremaster microcirculatory preparation has been extensively used in investigations of microvascular flow parameters, leukocyte recruitment and inflammatory response in sickle mice as described in previous studies (Kaul et al. J Clin Invest 96:2845-2853, 1996; Kaul and Hebbel, J Clin Invest 106:411-420, 2000: Jang et al. J Clin Invest 121:1397-1401, 2011; all incorporated here by reference with regard to the methods used). Soluble vascular cell adhesion molecule-1 (VCAM-1) was measured in EDTA plasma using mouse specific Quantikine ELISA kit (R&D Systems, Minneapolis, Minn., USA).

### Results:

### a) Marked Inhibition of Leukocyte Adherence and Emigration:

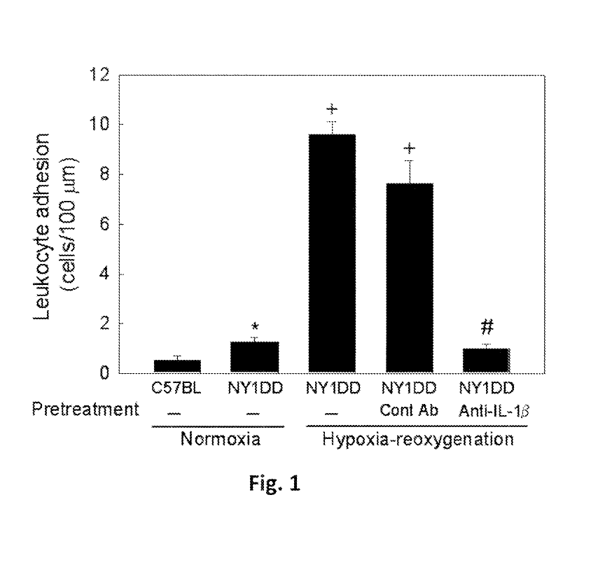

As depicted in Table 1 and FIG. 1, even in normoxic conditions NY1DD mice show >2-fold increase in leukocyte adhesion in venules compared with wild type mice (P<0.03). Hypoxia-reoxygenation in NY1DD mice (untreated or pretreated with irrelevant control antibody) caused marked inflammatory response as evidenced by ˜6- to 7.7 fold increase in leukocyte adhesion compared with normoxic NY1DD mice (P<0.0001). In contrast, NY1DD mice receiving a single i.p. injection of murine anti-IL-1β antibody prior to hypoxia-reoxygenation showed markedly alleviated leukocyte adhesion (P<0.0001 vs NY1DD mice subjected to hypoxia-reoxygenation or receiving control antibody); the resulting adhesion was not significantly different from that observed in normoxic C57BL and NY1DD mice.

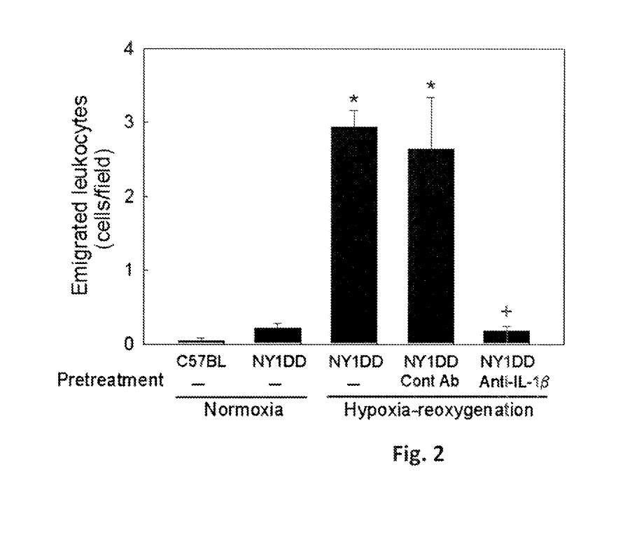

As shown in FIG. 2 and Table 1, hypoxia-reoxygenation in NY1DD mice (untreated or pretreated with control antibody) caused marked inflammatory response as evidenced by >13-fold increase in leukocyte extravasation within 3 hr of reoxygenation compared with normoxic NY1DD mice (P<0.00001). In contrast, NY1DD mice receiving a single i.p. injection of anti-IL-1β antibody prior to hypoxia showed marked alleviation of leukocyte emigration (P<0.0001) compared with NY1DD mice subjected to hypoxia-reoxygenation (untreated or pretreated with control antibody); the resulting leukocyte emigration was not significantly different from that observed in normoxic C57BL and NY1DD sickle mice.

TABLE 1
The Effect of anti-IL-1β antibody on leukocyte adhesion and trans-
endothelial emigration in Sickle (NY1DD) Mice Subjected to Hypoxia-Reoxygenation

| | Normoxia | | Hypoxia-Reoxygenation | | |
|---|---|---|---|---|---|
| | Wild type | | (Sickle mice) | | |
| Parameter | (C57BL mice) | Sickle mice | Untreated | Control Ab | Anti-IL-1β Ab |
| Diameter, | 28.7 ± 1.3 (22)* | 29.0 ± 0.8 (59) | 29.5 ± 0.6 (54) | 29.9 ± 1.7 (14) | 30.0 ± 0.7 (56) |
| μm | | | | | |
| Leukocyte | 0.5 ± 0.21 | 1.25 ± 0.18⁺ | 9.6 ± 0.5* | 7.6 ± 0.9* | 1.0 ± 0.17** |
| adhesion, | | | | | |
| cells/100 | | | | | |
| μm | | | | | |
| Leukocyte | 0.05 ± 0.05 | 0.22 ± 0.07 | 2.94 ± 0.23* | 3.0 ± 0.8* | 0.18 ± 0.07** |
| emigration, | | | | | |
| cells/field | | | | | |

Values are mean ± SE.
*Numbers in parentheses represent the number of venues examined for leukocyte adhesion and emigration;
⁺P < 0.026 vs. normoxic wild type controls;
*P < 0.0001 vs. respective normoxic values for NY1DD mice;
**P < 0.0001 vs. vs. NY1DD mice subjected to hypoxia-reoxygenation (untreated or receiving control isotype antibody [control Ab]).

### b) Anti-IL-1β Antibody Markedly Improves Micro-Hemodynamic Parameters:

Table 2 shows the effect of murine IL-1β antibody on micro-hemodynamic parameters in transgenic sickle (NY 1DD) mice subjected to hypoxia re-oxygenation. Under normoxic conditions. NY1DD mice showed almost 60% decrease in red cell velocity (Vrbc), wall shear rates and volumetric flow (Q) compared with wild type (C57BL) mice (each P<0.0001). Hypoxia-re-oxygenation caused further significant decline in Vrbc, wall shear rates and Q in untreated NY1DD mice subjected to hypoxia-reoxygenation compared with normoxic NY1DD mice (each P<0.0001). NY1DD mice pretreated with control antibody and subjected to hypoxia-re-oxygenation showed marked decrease in Vrbc and wall shear rates compared with normoxic NY1DD mice (P<0.007 and P<0.001, respectively). In contrast, all micro-hemodynamic parameters showed marked improvement in NY1DD mice treated with anti-IL-1β antibody compared with the hypoxia-reoxygenation groups (P<0.001-0.0001); the resulting values were not significantly different from normoxic wild-type (C57BL) mice. Importantly, frequent flow stasis observed in sickle mice subjected to hypoxia-reoxygenation was completely abolished by the anti-IL-1β antibody.

TABLE 2
Anti-1L-1β antibody improves Hemodynamic Parameters in
Venules of Sickle (NY1DD) Mice Subjected to Hypoxia-Reoxygenation

| | Normoxia | | Hypoxia-Reoxygenation | | |
|---|---|---|---|---|---|
| | Wild type | | (Sickle mice) | | |
| Parameter | (C57BL mice) | Sickle mice | Untreated | Control Ab | Anti-IL-1β□ |
| Diameter, | 28.7 ± 1.3 (22)* | 29.0 ± 0.8 (59) | 29.5 ± 0.6 (54) | 29.9 ± 1.7 (14) | 30.0 ± 0.7 (56) |
| μm | | | | | |
| Red cell | 6.3 ± 0.6 | 3.0 ± 0.2⁺ | 1.7 ± 0.1** | 1.9 ± 0.14** | 5.0 ± 0.3^{#} |
| Velocity, | | | | | |
| mm/s | | | | | |
| Wall shear | 1083 ± 86 | 521 ± 29⁺ | 297 ± 19** | 318 ± 22^{#} | 862 ± 73^{#} |
| rate, s⁻¹ | | | | | |
| Volumetric | 2.9 ± 0.48 | 1.3 ± 0.13⁺ | 0.75 ± 0.06** | 0.9 ± 0.13 | 2.3 ± 0.18^{#} |
| flow (Q), | | | | | |
| nl/s | | | | | |

Values are mean ± SE.
*Numbers in parentheses represent the number of venules examined for hemodynamic parameters;
⁺P < 0.0001 vs. normoxic wild type controls;
**P < 0.0001 vs. normoxic values for NY1DD mice;
^{#}P < 0.005-0.0001 vs. NY1DD mice subjected to hypoxia-reoxygenation (untreated or receiving control isotype antibody [control Ab]).

### c) Anti-1β Antibody Ameliorates Endothelial Oxidant Generation Caused by Hypoxia-Reoxygenation:

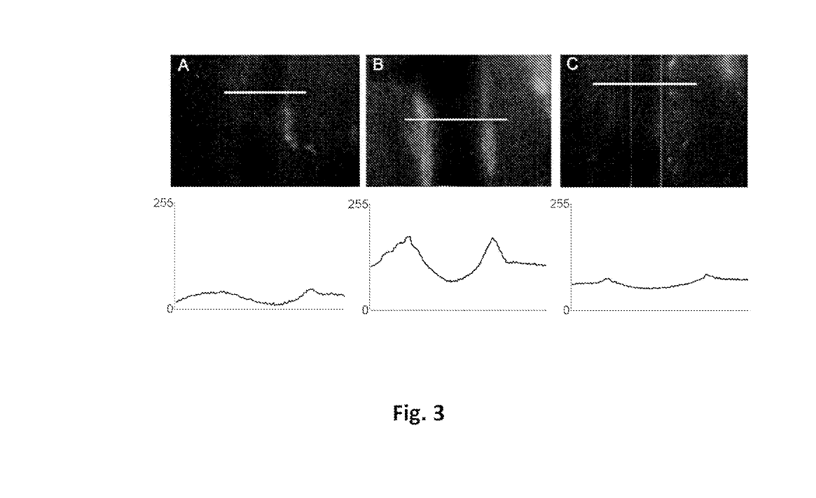

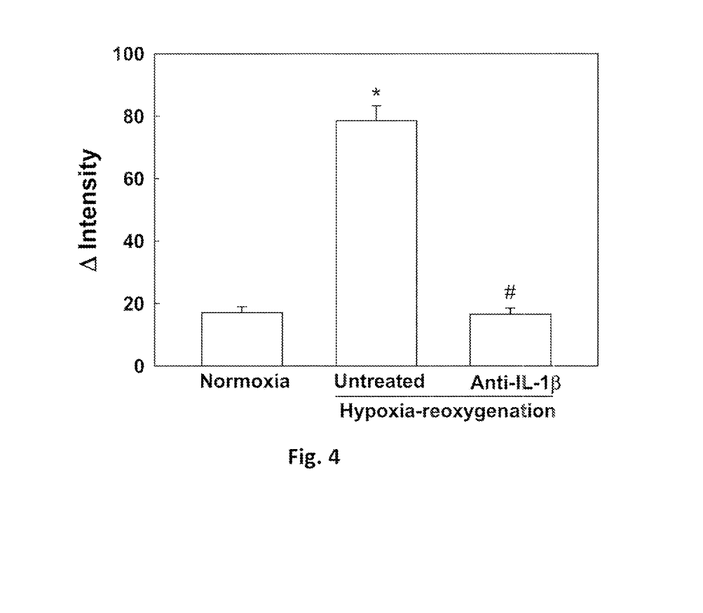

We monitored endothelial oxidant generation in cremaster venules by suffusing preparations with dihydrorhodamine-123 (DHR) and quantifying Δ intensity (Δ/) between the background and the venular endothelium. DHR is oxidized into fluorescent rhodamine after reaction with reactive oxygen species such as H₂O₂. The peak oxidant generation occurs after 30-45 min after reoxygenation. Hence, the endothelial oxidant generation was monitored 30 after reoxygenation. FIG. 3 shows fluorographs of cremaster muscle their corresponding DHR profiles in transgenic sickle (NY 1DD) mice. Hypoxia-reoxygenation alone caused almost 4.6-fold increase in Δ/ compared to normoxic NY1DD mice (P<0.0001) (FIG. 4). In contrast, pretreatment with anti-IL-1β antibody followed by hypoxia-reoxygenation caused marked attenuation of DHR fluorescence with Δ/ showing a decrease of almost 80% compared with untreated NY1DD mice subjected to hypoxia-reoxygenation alone (P<0.0001). Following the treatment with IL-1β, Δ/ was not significantly different from Δ/ levels for normoxic sickle mice.

d) sVCAM-1, an Endothelial Activation Marker:

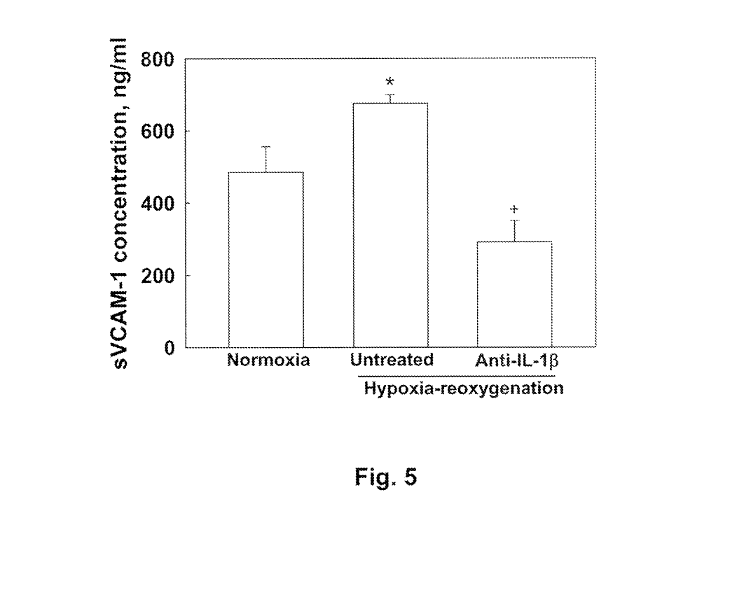

As shown in FIG. 5, hypoxia-reoxygenation caused almost 40% increase in plasma sVCAM-1 levels (ng/ml) in NY1DD mice compared with normoxic values (P<0.05). Pretreatment with anti-IL-1β antibody resulted in marked 57% decrease in sVCAM-1 concentrations in these mice (P<0.003).

### Summary and Significance:

We have examined efficacy of murine anti-IL-1β antibody (Novartis Pharma AG, Basel, Switzerland) in transgenic sickle (NY1DD) and transgenic-knockout sickle (BERK) mice (data not shown). Whereas BERK mice (C. Paszty et al., Science 278, 876, 1997) express exclusively human α- and β^{S}-globins, NY1DD mice express 56% human α and 75% β^{S} on a mouse homozygous β^{major} deletional background. For hypoxia-reoxygenation (H/R) experiments, we selected NY1DD mice based on their highly exaggerated response to H/R compared with C57BL mice NY1DD mice were subjected to 16 hr of hypoxia (8% O₂) followed by 3 hr of reoxygenation at ambient air. In NY1DD mice, hypoxia-reoxygenation (H/R) caused almost 8-fold increase in leukocyte adhesion and ˜13-fold increase in transendothelial leukocyte emigration in cremasteric venules as compared with normoxic NY1DD mice (each P<0.0001): leukocyte adhesion often resulted in flow blockage in post-capillary venules. In marked contrast. NY1DD mice, receiving a single i.p. injection of anti-IL-1β antibody (200 μg/mouse) 24 hrs prior to H/R, showed greater than 90% decrease in leukocyte adhesion and emigration compared with NY1DD mice subjected to H/R alone (P<0.0001); the resulting values were not significantly different from those for normoxic C57BL mice. Also, H/R caused 43% decline in venular wall shear rates in NY1DD mice compared with normoxic NY1DD mice (P<0.0001). In contrast, in NY1DD mice, pretreatment with the antibody followed by H/R almost completely normalized wall shear rates. Importantly, frequent flow stasis observed in NY1DD mice subjected to H/R was completely abolished by the anti-IL-1β antibody. Notably, the decrease in leukocyte adhesion in NY1DD mice receiving the antibody was associated with marked decrease in endothelial oxidant production as measured by dihydrorhodamine (DHR) oxidation intensity (P<0.0001), suggesting alleviation of endothelial activation. The reduced endothelial activation was further evident by 60% decrease in the serum levels of soluble vascular cell adhesion molecule-1 (P<0.016). In experiments with BERK mice, a single dose of anti-IL-1β antibody (300 μg) was injected i.p. under normoxic conditions. We did not subject the severe BERK model to H/R because of their low tolerance to hypoxia. In BERK mice, anti-IL-1β caused 41% decrease in leukocyte adhesion (P<0.036 vs. untreated group). The reduced leukocyte adhesion was accompanied by a pronounced 3.5-fold increase in wall shear rates (P<0.0001), suggesting a generalized improvement in the microvascular flow.

The experiments provide a strong basis for therapeutic application of anti IL-1β antibody in the prevention of complications (e.g., endothelial activation and leukocyte recruitment caused by inflammation, infection or hypersensitivity reactions) that can lead to intravascular sickling and vaso-occlusion in sickle cell disease. The ameliorating effect of the Novartis anti-IL-1β antibody may be attributed its ability to block IL-1β-dependent activation of NF-κB and thereby inhibit upregulation of endothelial adhesion molecules. Based on the long half-life in vivo and anti-inflammatory efficacy of Novartis anti-IL-1β□ canakinumab antibody (Ilaris®), our results with the corresponding mouse antibody provide a strong basis for therapeutic application of canakinumab in the (especially preventative) clinical management of sickle cell disease.

### Example 2

### Clinical trial with canakinumab

In order to asses the suitability of an IL-1beta Compound, canakinumab (Novartis AG, Basle, Switzerland), a placebo-controlled clinical trial in children or adults to assess the clinical efficacy, safety, pharmacokinetics and pharmacodynamics in patients with Sickle Cell Disease is conducted.

Patients are administered a single injection of canakinumab (e.g. 50, 100, 150 or 300 mg s.c. for adults, in children with body weight below 40 kg e.g. 4 mg/kg). Clinical response is measured by improvement of symptoms (e.g., pain, fever, patient global assessment) or by preventing or alleviating inflammatory and painful episodes characteristic for Sickle Cell Disease or by improving patient global assessment. Prevention of disease exacerbations is measured by the frequency of episodes in the placebo arm vs. the active treatment arm within a predefined time period, e.g. 2 months, 4 months, or 6 months.

Patients could also be dosed according to a dosing scheme every month, every second month, every third month, fourth month, fifth month, 2 times per year, or yearly.

### Example 3

### Relevant Sequences of Canakinumab

| Canakinumab Heavy chain variable region | |
|---|---|
| (amino acid sequence SEQ ID NO: 1) | |
| TCAG | |
| Q - | - 1 |
| GTGCAGCTGGTGGAGTCTGGGGGAGGCGTGGTCCAGCCTGGGAGGTCCCTGAGACTCTCC | |
| V Q L V E S G G G V V Q P G R S L R L S | - 21 |
| TGTGCAGCGTCTGGATTCACCTTCAGTGTTTATGGCATGAACTGGGTCCGCCAGGCTCCA | |
| C A A S G F T F S V Y G M N W V R Q A P | - 41 |
| GGCAAGGGGCTGGAGTGGGTGGCAATTATTTGGTATGATGGAGATAATCAATACTATGCA | |
| G K G L E W V A I I W Y D G D N Q Y Y A | - 61 |
| GACTCCGTGAAGGGCCGATTCACCATCTCCAGAGACAATTCCAAGAACACGCTGTATCTG | |
| D S V K G R F T I S R D N S K N T L Y L | - 81 |
| CAAATGAACGGCCTGAGAGCCGAGGACACGGCTGTGTATTATTGTGCGAGAGATCTTAGG | |
| Q M N G L R A E D T A V Y Y C A R D L R | - 101 |
| ACTGGGCCTTTTGACTACTGGGGCCAGGGAACCCTGGTCACCGTCTCCTC | |
| T G P F D Y W G Q G T L V T V S S | 118 |
| Canakinumab Light chain variable region | |
| (amino acid sequence SEQ ID NO: 2 | |
| TGAA | |
| E | - 1 |
| ATTGTGCTGACTCAGTCTCCAGACTTTCAGTCTGTGACTCCAAAGGAGAAAGTCACCATC | |
| I V L T Q S P D F Q S V T P K E K V T I | - 21 |
| ACCTGCCGGGCCAGTCAGAGCATTGGTAGTAGCTTACACTGGTACCAGCAGAAACCAGAT | |
| T C R A S Q S I G S S L H W Y Q Q K P D | - 41 |
| CAGTCTCCAAAGCTCCTCATCAAGTATGCTTCCCAGTCCTTCTCAGGGGTCCCCTCGAGG | |
| Q S P K L L I K Y A S Q S F S G V P S R | - 61 |
| TTCAGTGGCAGTGGATCTGGGACACATTTCACCCTCACCATCAATAGCCTGGAAGCTGAA | |
| F S G S G S G T D F T L T I N S L E A E | - 81 |
| GATGCTGCAGCGTATTACTGTCATCAGAGTAGTAGTTTACCATTCACTTTCGGCCCTGGG | |
| D A A A Y Y C H Q S S S L P F T F G P G | - 101 |
| ACCAAAGTGGATATCAAA | - 107 |
| T K V D I K | |

1. A method of using a preventatively effective amount of an IL-1β-ligand/IL-1 receptor disrupting compound for the prevention of one or more manifestations or complications that can lead to intravascular sickling and vaso-occlusion in sickle cell disease in a patient in need of such use.

2. The method according to claim 1, where the manifestation or complication is based on or is endothelial activation and/or leukocyte recruitment.

3. The method according to claim 1, where the manifestation or complication is one or more selected from the group consisting of ischemia reperfusion injury, acute chest syndrome, especially acute chest injury, hyposplenism, asplenism, cardiovascular damages or another manifestation of long term SCD where caused by inflammation, infection or hypersensitivity reactions.

4. The method according to claim 1, where the patient to be treated is an individual with homozygosity for the mutation that causes hemoglobin S (HbS), a heterozygote with sickle-beta-thalassemia with an SCD supporting combination of HbS gene and beta-thal gene, an individual with one sickle cell gene and one null alleleor an individual with hemoglobin SC disease.

5. The method according to claim 1, where the IL-1β-ligand/IL-1 receptor disrupting compound is selected from small molecular compounds disrupting IL-1β ligand-IL-1 receptor interaction, IL-1β antibodies or IL-1 receptor antibodies, e.g. IL-1β binding molecules described herein, e.g. antibodies disclosed herein, e.g. IL-1β binding compounds or IL-1 receptor binding compounds, and RNA compounds decreasing either IL-1β ligands or IL-1 receptor protein levels.

6. The method according to claim 5 wherein the IL-1β-ligand/IL-1 receptor disrupting compound is an IL-1β binding molecule which comprises an antigen binding site comprising at least one immunoglobulin heavy chain variable domain (V_{H}) which comprises in sequence hypervariable regions CDR1, CDR2 and CDR3, said CDR1 having the amino acid sequence Val-Tyr-Gly-Met-Asn (SEQ ID NO: 3), said CDR2 having the amino acid sequence Ile-Ile-Trp-Tyr-Asp-Gly-Asp-Asn-Gln-Tyr-Tyr-Ala-Asp-Ser-Val-Lys-Gly (SEQ ID NO: 4), and said CDR3 having the amino acid sequence Asp-Leu-Arg-Thr-Gly-Pro (SEQ ID NO: 5); and direct equivalents thereof, and at least one immunoglobulin light chain variable domain (V_{L}) which comprises in sequence hypervariable regions CDR1′, CDR2′ and CDR3′, said CDR1′ having the amino acid sequence Arg-Ala-Ser-Gln-Ser-Ile-Gly-Ser-Ser-Leu-His (SEQ ID NO: 6), said CDR2′ having the amino acid sequence Ala-Ser-Gln-Ser-Phe-Ser (SEQ ID NO: 7) and said CDR3′ having the amino acid sequence His-Gln-Ser-Ser-Ser-Leu-Pro (SEQ ID NO: 8); and direct equivalent thereof: or for each of the sequences a homologous variant having at least 80%, 82%, 84%, 86%, 88%, 90%, 92%, 94%, 96% or 98% homology.

7. The method according to claim 6, wherein the IL-1β-ligand/IL-1 receptor disrupting compound is an antibody where the both heavy and light chains are of human origin.

8. The method according to claim 7, wherein in the antibody the heavy and light chain framework regions have the amino acid sequence according to SEQ ID NO: 1 for the heavy chain regions, in sequence. FR1, FR2, FR3 and FR4 and the amino acid sequence according to SEQ ID NO: 2 for the light chain regions, in sequence, FR1′, FR2′, FR3′ and FR4′.

9. The method according to claim 1 wherein the IL-1β-ligand/IL-1 receptor disrupting compound has a K_{D} for binding to IL-1β of about 10 nM, 1 nM, preferably 100 pM, more preferably 50 pM or less.

10. An IL-1β-ligand/IL-1 receptor disrupting compound for use in a method according to claim 1.

11. (canceled)

12. (canceled)

13. (canceled)

14. (canceled)


SEQUENCE LISTING (8 sequences)

SEQ ID NO: 1 - protein, 118 aa, homo sapiens [sequence omitted]

SEQ ID NO: 2 - protein, 107 aa, homo sapiens [sequence omitted]

SEQ ID NO: 3 - protein, 5 aa, HOMO SAPIENS
VYGMN

SEQ ID NO: 4 - protein, 17 aa, HOMO SAPIENS
IIWYDGDNQYYADSVKG

SEQ ID NO: 5 - protein, 6 aa, HOMO SAPIENS
DLRTGP

SEQ ID NO: 6 - protein, 11 aa, HOMO SAPIENS
RASQSIGSSLH

SEQ ID NO: 7 - protein, 6 aa, HOMO SAPIENS
ASQSFS

SEQ ID NO: 8 - protein, 7 aa, HOMO SAPIENS
HQSSSLP